(19) United States
(12) Patent Application Publication
(10) Pub. No.: US 2023/0238149 A1
(43) Pub. Date: Jul. 27, 2023

(54) IMAGE PROCESSING APPARATUS, IMAGE PROCESSING METHOD, AND NON-TRANSITORY STORAGE MEDIUM

(71) Applicant: NEC Corporation, Minato-ku, Tokyo (JP)

(72) Inventors: Gaku Nakano, Tokyo (JP); Shoji Nishimura, Tokyo (JP); Yoshitaka Higeta, Tokyo (JP); Youtarou Noda, Tokyo (JP); Shigeki Shinoda, Tokyo (JP); Yuzo Senda, Tokyo (JP); Shin Tominaga, Tokyo (JP); Masumi Ishikawa, Tokyo (JP); Daisuke lkefuji, Tokyo (JP); Kana Takizawa, Tokyo (JP); Kenichi lshi, Toky (JP); Yoshiyuki Satou, Tokyo (JP); Eishi Arita, Tokyo (JP); Shota Yammoto, Tokyo (JP); Shinya Yamasaki, Tokyo (JP); Narumitsu Notomi, Tokyo (JP); Aki Nakamura, Tokyo (JP)

(73) Assignee: NEC Corporation, Minato-ku, Tokyo (JP)

(21) Appl. No.: 18/007,996

(22) PCT Filed: Mar. 8, 2021
(86) PCT No.: PCT/JP2021/008882

§ 371 Date: Dec. 2, 2022

(30) Foreign Application Priority Data
Jun. 5, 2020 (JP) 2020-098403

(51) Int. Cl. G16H 50/80; G16H 50/30; G06V 10/74; G06F 3/01

(52) U.S. Cl. CPC G16H 50/80; G16H 50/30; G06V 10/761; G06F 3/013; G06V 2201/07

(57) ABSTRACT

The present invention provides an image processing apparatus (10) including: a companion determination unit (12) that determines a companion of each of a plurality of persons detected from an image, based on the image; a distance computation unit (13) that computes a first distance being a distance to the nearest person among the persons other than a companion, for the each person; and a risk information generation unit (14) that generates, with use of the first distance, infection risk information being information related to a risk of getting an infectious disease or a safety rate of not getting an infectious disease within a target region being a region included in the image.

### TECHNICAL FIELD

The present invention relates to an image processing apparatus, an image processing method, and a program.

### BACKGROUND ART

In recent years, image processing is used for various purposes. For example, Patent Document 1 describes a system in which a reaching range of a droplet from a first target person is deviated from a respiratory area of a second target person by adjusting an environment within a space, wherein a position and an orientation of a face of each of the first target person and the second target person are determined by image processing.

### RELATED DOCUMENT

### Patent Document

[Patent Document 1] International Publication No. WO2020/044826

### DISCLOSURE OF THE INVENTION

### Technical Problem

In order to suppress spread of an infectious disease, it is preferable to appropriately take a measure for securing a person-to-person distance, such as, for example, “securing an interval between seats”, “limiting the number of visitors”, or “increasing a distance between persons in line” at a place (such as a store, an institution, or a facility) where people are gathering. Recognizing a place where such a measure is not appropriately taken enables to take various measures such as “avoiding the place”, “encouraging improvement for the place”, “ceasing operation at the place” for suppressing spread of an infectious disease.

Meanwhile, it is difficult to thoroughly take a measure for securing a distance also for a group such as family members or lovers under authority of an administrator of each place. Therefore, it is not reasonable to evaluate that a measure for suppressing spread of an infectious disease is not appropriately taken at such a place, based on that a distance between companions is not secured.

In view of circumstances as described above, it is difficult to recognize whether a measure for suppressing spread of an infectious disease is appropriately taken at each place. One of objects of the present invention is to make it easy to recognize whether a measure for suppressing spread of an infectious disease is appropriately taken at each place.

### Solution to Problem

The present invention provides an image processing apparatus including:

a companion determination unit that determines a companion of each of a plurality of persons detected from an image, based on the image;

a distance computation unit that computes a first distance being a distance to a nearest person among the persons other than a companion, for the each person; and

a risk information generation unit that generates, with use of the first distance, infection risk information being information related to a risk of getting an infectious disease or a safety rate of not getting an infectious disease within a target region being a region included in the image.

Further, the present invention provides an image processing method including,

by a computer:

determining a companion of each of a plurality of persons detected from an image, based on the image;

computing a first distance being a distance to a nearest person among the persons other than a companion, for the each person; and

generating, with use of the first distance, infection risk information being information related to a risk of getting an infectious disease or a safety rate of not getting an infectious disease within a target region being a region included in the image.

Further, the present invention provides a program causing a computer to function as:

a companion determination unit that determines a companion of each of a plurality of persons detected from an image, based on the image;

a distance computation unit that computes a first distance being a distance to a nearest person among the persons other than a companion, for the each person; and

a risk information generation unit that generates, with use of the first distance, infection risk information being information related to a risk of getting an infectious disease or a safety rate of not getting an infectious disease within a target region being a region included in the image.

### Advantageous Effects of Invention

The present invention makes it easy to recognize whether a measure for suppressing spread of an infectious disease is appropriately taken at each place.

### BRIEF DESCRIPTION OF THE DRAWINGS

FIG. 1 is a diagram illustrating a usage environment of an image processing apparatus according to an example embodiment.

FIG. 2 is a diagram illustrating one example of a functional configuration of the image processing apparatus.

FIG. 3 is a diagram illustrating one example of information stored in a storage unit.

FIG. 4 is a diagram illustrating a hardware configuration example of the image processing apparatus.

FIG. 5 is a flowchart illustrating one example of processing to be performed by the image processing apparatus.

FIG. 6 is a diagram illustrating one example of a first distance computation method to be performed in step S20 in FIG. 5.

FIG. 7 is a diagram illustrating one example of an image to be displayed on a display unit in step S40 in FIG. 5.

FIG. 8 is a diagram illustrating one example of an image to be displayed on the display unit in step S40 in FIG. 5.

FIG. 9 is a flowchart illustrating one example of processing to be performed by the image processing apparatus.

FIG. 10 is a flowchart illustrating one example of processing to be performed by the image processing apparatus.

FIG. 11 is a diagram illustrating one example of an image to be displayed in step S40 in FIG. 5.

FIG. 12 is a diagram illustrating one example of an image to be displayed in step S40 in FIG. 5.

### DESCRIPTION OF EMBODIMENTS

Hereinafter, an example embodiment according to the present invention is described with reference to the drawings. Note that, in all drawings, a similar constituent element is indicated by a similar reference sign, and description thereof is omitted as necessary.

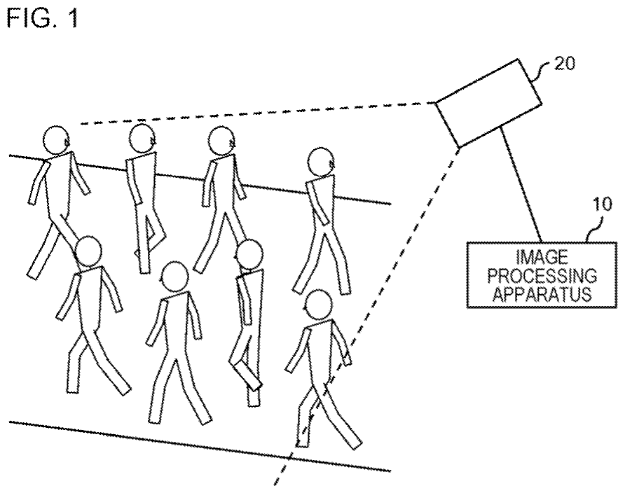

FIG. 1 is a diagram illustrating a usage environment of an image processing apparatus 10 according to the example embodiment. The image processing apparatus 10 is used together with an image capturing apparatus 20.

The image capturing apparatus 20 is, for example, a fixed camera, and repeatedly photographs a region (hereinafter, described as a target region) where a plurality of persons, for example, an unspecified large number of persons come and go. Therefore, an image to be generated by the image capturing apparatus 20 includes a plurality of persons. The image capturing apparatus 20 may also be a surveillance camera installed, for example, in a store, an institution, a facility, or the like. A frame rate of an image to be generated by the image capturing apparatus 20 is optional, but may be, for example, a frame rate at which a moving image is composed. As one example, the image capturing apparatus 20 is communicably connected to the image processing apparatus 10, and can transmit a generated image to the image processing apparatus 10.

The image processing apparatus 10 computes, by processing an image generated by the image capturing apparatus 20, a distance between persons within a target region, specifically, a distance (hereinafter, described as a first distance) between a certain person (hereinafter, described as a person as a reference), and a person nearest to the certain person. Further, the image processing apparatus 10 generates, with use of the first distance, information (hereinafter, infection risk information) related to a risk of getting an infectious disease or a safety rate of not getting an infectious disease within a target region. The infection risk information becomes an index as to whether a measure for suppressing spread of an infectious disease is appropriately taken within the target region.

In the example illustrated in FIG. 1, the image processing apparatus 10 is connected to one image capturing apparatus 20. However, the image processing apparatus 10 may be connected to a plurality of image capturing apparatuses 20. In this case, the plurality of image capturing apparatuses 20 capture target regions different from each other. Further, each of the plurality of image capturing apparatuses 20 transmits an image to the outside in association with information (hereinafter, described as image capturing apparatus identification information) for identifying the image capturing apparatus 20. This enables to easily generate infection risk information for each of a plurality of, for example, 100 or more large number of target regions.

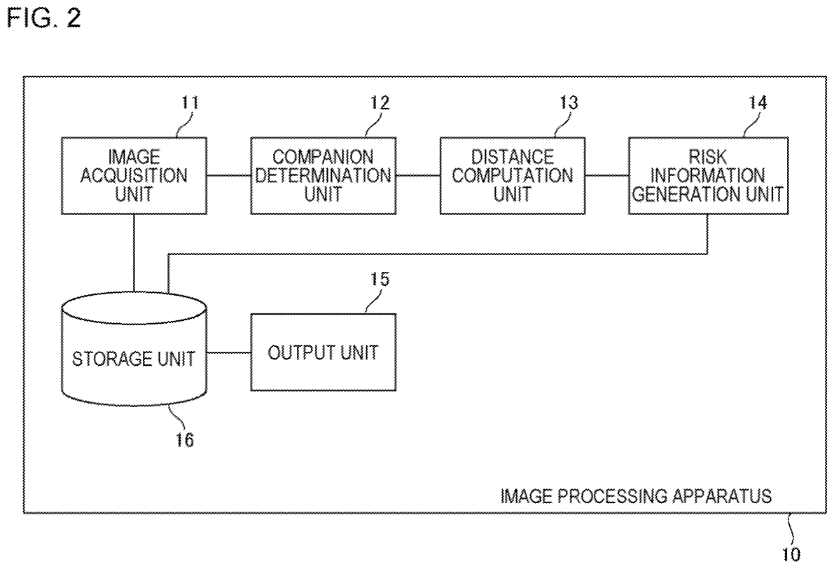

FIG. 2 is a diagram illustrating one example of a functional configuration of the image processing apparatus 10. The image processing apparatus 10 illustrated in FIG. 2 includes an image acquisition unit 11, a companion determination unit 12, a distance computation unit 13, a risk information generation unit 14, an output unit 15, and a storage unit 16. Note that, the image processing apparatus 10 may not include the storage unit 16. In this case, an external apparatus configured to be communicable with the image processing apparatus 10 includes the storage unit 16.

The image acquisition unit 11 acquires an image including a plurality of persons generated by the image capturing apparatus 20. The image acquisition unit 11 causes the storage unit 16 to store an acquired image. In a case where the image processing apparatus 10 and the image capturing apparatus 20 are communicably connected to each other, the image acquisition unit 11 can receive an image from the image capturing apparatus 20. In addition to the above, an image generated by the image capturing apparatus 20 may be stored in any storage apparatus. Further, the image acquisition unit 11 may acquire an image stored in the storage apparatus at any timing.

The image acquisition unit 11 may acquire an image generated by the image capturing apparatus 20 by batch processing, or may acquire by real-time processing. In a case of performing acquisition and analyzation of an image by batch processing, it is possible to comprehensively evaluate whether a measure for suppressing spread of an infectious disease is appropriately taken within each target region, based on an image for a predetermined time period (example: for one hour, for one day, for one week, or the like). On the other hand, in a case of performing acquisition and analyzation of an image by real-time processing, it is possible to evaluate whether a measure for suppressing spread of an infectious disease is appropriately taken within each target region at a timing at which a latest image is acquired.

The companion determination unit 12 determines, by processing an image acquired by the image acquisition unit 11, a companion of each of a plurality of persons detected from the image. Since a technique for detecting a person from an image is widely known, description thereof is omitted herein. Hereinafter, one example of a method of determining a companion of each person is described.

### First Example

The companion determination unit 12 determines, based on a duration of a time of a state in which a distance between any person (hereinafter, described as a first person) and another person among a plurality of detected persons is equal to or less than a threshold value, a companion of the first person. Specifically, the companion determination unit 12 determines that another person whose duration of time is equal to or more than a threshold value is a companion of the first person. A specific example of a distance computation method based on an image is described later.

### Second Example

The companion determination unit 12 determines a companion of the first person, based on lines of sight of the first person and another person among a plurality of detected persons. Specifically, the companion determination unit 12 determines a companion of the first person, based on at least either one of a duration of time of a line-of-sight state in which lines of sight of the first person and another person are directed toward each other (looking at each other), and a frequency of occurrence of the line-of-sight state within a predetermined time. The frequency is “the number of times”, “a ratio of a time during which the line-of-sight state is attained with respect to a predetermined time”, or the like.

More specifically, the companion determination unit 12 determines that another person whose duration of time of a line-of-sight state in which lines of sight of the first person and the another person are directed toward each other (looking at each other) is equal to or more than a threshold value is a companion of the first person. Further, the companion determination unit 12 determines that another person whose frequency of occurrence of the line-of-sight state with respect to the first person is equal to or more than a threshold value within a predetermined time is a companion of the first person.

### Third Example

The companion determination unit 12 determines a companion of the first person, based on a physical contact status of the first person and another person among a plurality of detected persons. For example, the companion determination unit 12 may determine that another person who satisfies one or any plurality of the following conditions is a companion of the first person.
A physical contact has occurred with the first person.
A frequency of a physical contact with the first person is equal to or more than a threshold value. The frequency is “the number of times”, “a ratio of a physical contact time with respect to a predetermined time”, or the like.
A duration of time of a physical contact state with the first person is equal to or more than a threshold value.
A physical contact of a predetermined pattern, which is defined in advance, has occurred with the first person. The predetermined pattern is an action which may occur between companions, such as “holding hands”, “putting an arm around another person's shoulder”, “putting an arm around another person's waist”, “carrying in arm, “riding on one's shoulders”, “carrying on one's back”, or the like.

### Fourth Example

In addition to determination references of the first to third examples, the companion determination unit 12 determines a companion of the first person, based on attributes of the first person and another person among a plurality of detected persons. The attribute is estimable from an image, and is such as, for example, “a type being selected from an adult, a child, and an old person”, “age”, and “gender”. The companion determination unit 12 determines that another person who satisfies determination references of the first to third examples, and satisfies a condition regarding an attribute relationship with respect to the first person is a companion of the first person. For example, in a case where the first person is an “adult”, a condition of a companion is a “child”.

### Fifth Example

The companion determination unit 12 determines that another person who satisfies any plurality of determination references among determination references of the first to fourth examples is a companion of the first person.

The distance computation unit 13 computes the first distance being a distance to a nearest person among persons other than a companion, for each detected person. A specific example of a first distance computation method is described later.

The risk information generation unit 14 generates, with use of the first distance, infection risk information within a target region being a photographing target of the image capturing apparatus 20. As one example, the risk information generation unit 14 determines whether the first distance is equal to or less than a reference value, and generates infection risk information by using the determination result. The reference value is defined based on a so-called social distance. The social distance is a physical distance that should be secured between persons adjacent to each other in order to prevent infection of an infectious disease. Further, magnitude of the reference value is set based on a main infection route of a target infectious disease. For example, a value being equal to or more than 1.5 m and being equal to or less than 6 m is used as a reference value for an infectious disease in which droplet infection is a main cause. Further, regarding an infectious disease in which contact infection is a main cause, a value being equal to or more than 50 cm and being equal to or less than 1.5 m is used as a reference value. The risk information generation unit 14 can store, in the storage unit 16, generated infection risk information in association with information indicating each target region. The storage unit 16 may further store each target region and information for identifying a place (such as a store, an institution, or a facility) including each target region in association with each other.

Note that, infection risk information indicates, for example, a risk of getting an infectious disease or a safety rate of not getting an infectious disease within a target region. Such infection risk information becomes an index as to whether a measure for suppressing spread of an infectious disease is appropriately taken within the target region. For example, the following methods are cited as a method of generating infection risk information from the above-described determination result.

### (Method 1)

The risk information generation unit 14 computes, for each image, the number of combinations of persons in which the first distance becomes equal to or less than a reference value, and increases a risk indicated by infection risk information, as the number increases. Using this method enables the risk information generation unit 14 to generate infection risk information for each image. Specifically, it is possible to evaluate, based on a content represented by each image, whether a measure for suppressing spread of an infectious disease is appropriately taken at a point of time when each image is generated.

### (Method 2)

The risk information generation unit 14 derives the number of combinations of persons in which the first distance becomes equal to or less than a reference value, for each image group for a unit time period, and increases a risk indicated by infection risk information, as a statistical value (such as an average value, a maximum value, a minimum value, a mode, or a median) thereof increases. Using this method enables to comprehensively evaluate whether a measure for suppressing spread of an infectious disease is appropriately taken within each target region, based on the entirety of a plurality of images generated for a predetermined time period (example: thirty minutes, one hour, one day, or one week).

### (Method 3)

The risk information generation unit 14 derives the number of combinations of persons in which the first distance becomes equal to or less than a reference value, for each image group for a unit time period, and increases a risk indicated by infection risk information, as the most recent number per unit time increases. Using this method enables to evaluate whether a measure for suppressing spread of an infectious disease is appropriately taken at a point of time when the image group is generated, based on a content represented by a predetermined number of most recent images.

### (Method 4)

The risk information generation method 14 derives “the number of combinations of persons in which a state that the first distance is equal to or less than a reference value is continued for a reference period of time or longer”, in place of “the number of combinations of persons in which the first distance becomes equal to or less than a reference value” in the method 2. Further, it is assumed that other conditions are similar to those of the method 2. Using this method enables to comprehensively evaluate whether a measure for suppressing spread of an infectious disease is appropriately taken within each target region, based on the entirety of a plurality of images generated for a predetermined time period (example: thirty minutes, one hour, one day, or one week).

### (Method 5)

The risk information generation unit 14 derives “the number of combinations of persons in which a state that the first distance is equal to or less than a reference value is continued for a reference period of time or longer”, in place of “the number of combinations of persons in which the first distance becomes equal to or less than a reference value” in the method 3. Further, it is assumed that other conditions are similar to those of the method 3. Using this method enables to evaluate whether a measure for suppressing spread of an infectious disease is appropriately taken at a point of time when the image group is generated, based on a content represented by a predetermined number of most recent images.

### (Method 6)

In the methods 2 to 5, the risk information generation unit 14 computes the above-described number of combinations per unit time and per unit area, and increases a risk indicated by infection risk information, as the number increases.

Note that, in the methods 2 to 6, the risk information generation unit 14 uses a processing result on a plurality of images generated at a different timing. Further, it is possible to determine a same person being present redundantly within different images by using a feature value (such as face information) of an external appearance or position information of a person, and identify a combination with respect to an already detected person and a combination with respect to a newly detected person from each other.

Note that, the risk information generation unit 14 may use, as infection risk information, a fact as it is in which the first distance becomes equal to or less than a reference value.

The output unit 15 outputs various pieces of information stored in the storage unit 16. The output unit 15 may display infection risk information on a display apparatus such as a display or a projection apparatus. In addition to the above, the output unit 15 may store the infection risk information in a predetermined web server, and allow an unspecified large number of persons to be accessible via a communication network such as the Internet. Another example of information to be output from the output unit 15 is described later.

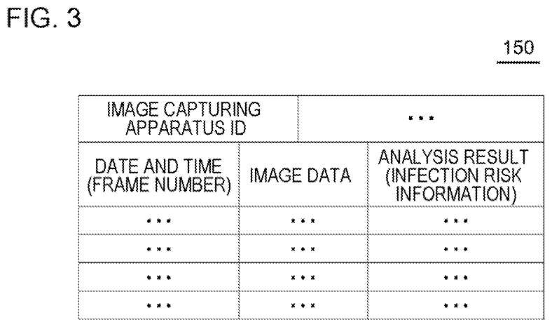

FIG. 3 is a diagram illustrating one example of information stored in the storage unit 16. In the example illustrated in FIG. 3, the storage unit 16 stores an image (described as image data in FIG. 3) generated by the image capturing apparatus 20 in association with information (e.g., a date and time itself, or a frame number) for determining a date and time when the image is generated. Further, the storage unit 16 stores an image generated by the image capturing apparatus 20 together with information (described as an analysis result in FIG. 3) acquired by processing the image. Note that, the analysis result may include infection risk information.

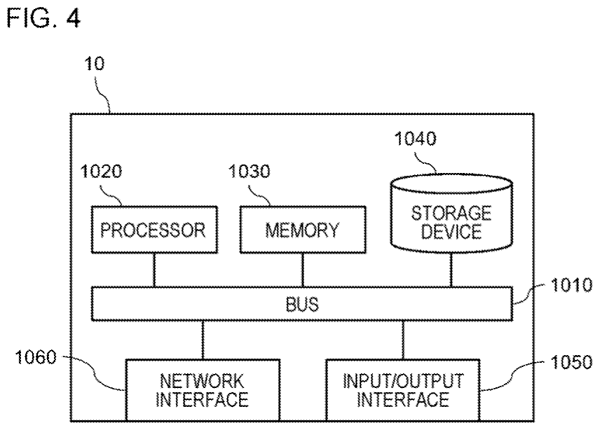

FIG. 4 is a diagram illustrating a hardware configuration example of the image processing apparatus 10. The image processing apparatus 10 includes a bus 1010, a processor 1020, a memory 1030, a storage device 1040, an input/output interface 1050, and a network interface 1060.

The bus 1010 is a data transmission path along which the processor 1020, the memory 1030, the storage device 1040, the input/output interface 1050, and the network interface 1060 mutually transmit and receive data. However, a method of mutually connecting to the processor 1020 and the like is not limited to bus connection.

The processor 1020 is a processor to be achieved by a central processing unit (CPU), a graphics processing unit (GPU), or the like.

The memory 1030 is a main storage apparatus to be achieved by a random access memory (RAM) or the like.

The storage device 1040 is an auxiliary storage apparatus to be achieved by a hard disk drive (HDD), a solid state drive (SSD), a memory card, a read only memory (ROM), or the like. The storage device 1040 stores a program module achieving each function (e.g., the image acquisition unit 11, the companion determination unit 12, the distance computation unit 13, the risk information generation unit 14, and the output unit 15) of the image processing apparatus 10. The processor 1020 achieves each function associated with the program module by reading each program module in the memory 1030 and executing each program module. Further, the storage device 1040 also functions as the storage unit 16.

The input/output interface 1050 is an interface for connecting the image processing apparatus 10 and various pieces of input/output equipment with each other.

The network interface 1060 is an interface for connecting the image processing apparatus 10 to a network. The network is, for example, a local area network (LAN) or a wide area network (WAN). A method of connecting the network interface 1060 to a network may be wireless connection, or may be wired connection. The image processing apparatus 10 may communicate with the image capturing apparatus 20 via the network interface 1060.

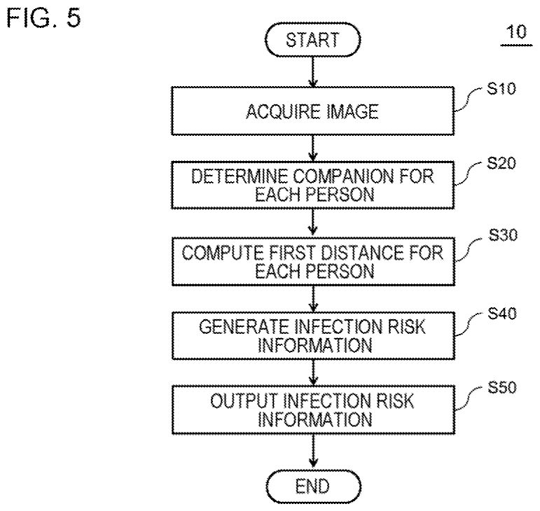

FIG. 5 is a flowchart illustrating a first example of processing to be performed by the image processing apparatus 10. Hereinafter, a case where an image is acquired and analyzed by batch processing, and a case where an image is acquired and analyzed by real-time processing are described separately.

### (Batch Processing)

First, the image acquisition unit 11 acquires a plurality of images for a predetermined time period (example: thirty minutes, one hour, one day, one week, or the like) being a processing target (S10). Next, the companion determination unit 12 analyzes the plurality of images for the predetermined time period, detects a person included within the images, and also determines a companion for each person (S20). Then, the companion determination unit 12 registers information indicating a companion in association with each of the detected persons. The companion determination unit 12 can determine a same person being present redundantly within different images by using, for example, a feature value of an external appearance or position information of a person. Then, the companion determination unit 12 can identify the plurality of detected persons from one another by using, for example, the feature value of the external appearance. Further, the companion determination unit 12 can determine a companion of each person, for example, based on any of the above-described first to fifth examples.

When the companion determination unit 12 finishes an analysis of all of the plurality of images for the predetermined time period, the distance computation unit 13 analyzes each of the plurality of images for the predetermined time period, and computes a distance (first distance) to a nearest person among the persons other than a companion, for each person (S30). The distance computation unit 13 can recognize a companion of each person, based on a determination result in S20. In computation in S30, the distance computation unit 13 computes the first distance by using a height and a position of a person being a distance computation target within an image, and an upward/downward orientation of the image capturing apparatus 20 that has generated the image. At this occasion, as will be described later in detail, the distance computation unit 13 uses a value (hereinafter, described as a reference height) set in advance as a height of a person.

When the distance computation unit 13 finishes an analysis of all of the plurality of images for the predetermined time period, the risk information generation unit 14 generates infection risk information by using the first distance generated in S30. The risk information generation unit 14 generates infection risk information, for example, based on any of the above-described methods 1 to 6. In a case of the example being batch processing, for example, the risk information generation unit 14 may adopt any of the methods 2, 4, and 6, and comprehensively evaluate whether a measure for suppressing spread of an infectious disease is appropriately taken within each target region, based on the entirety of a plurality of images generated for a predetermined time period. Note that, the risk information generation unit 14 may adopt any of the methods 1, 3, 5, and 6, and evaluate a status at each time.

Thereafter, the output unit 15 outputs infection risk information generated in S40 (S50). In this case, the output unit 15 may determine a target region where a risk of getting an infectious disease indicated by infection risk information is higher than a predetermined level (a target region where safety is lower than a predetermined level), and output information (such as a list of a determined target region) indicating the determined target region. Further, the output unit 15 may output information (such as a list of a determined place) indicating a place including the determined target region. As described above, it is possible to determine a place including a determined target region by associating a target region to be captured by each image capturing apparatus 20 and information for identifying a place (such as a store, an institution, or a facility) including each target region with each other, and using the information.

### (Real-Time Processing)

First, the image acquisition unit 11 acquires one image being a processing target (S10). Next, the companion determination unit 12 detects a person included within a newly acquired image, and also determines a companion for each person, based on the image or an image acquired so far (S20). Then, the companion determination unit 12 registers information indicating a companion in association with each of the detected persons. The companion determination unit 12 can determine a same person being present redundantly within different images by using, for example, a feature value of an external appearance or position information of a person. Then, the companion determination unit 12 can identify the plurality of detected persons from one another by using, for example, the feature value of the external appearance. Further, the companion determination unit 12 can determine a companion of each person, for example, based on any of the above-described first to fifth examples.

Subsequently, the distance computation unit 13 analyzes a newly acquired image, and computes a distance (first distance) to a nearest person among the persons other than a companion, for each person (S30). The distance computation unit 13 can recognize a companion of each person, based on a result determined in S20 up to the point of time. In computation in S30, the distance computation unit 13 computes the first distance by using a height and a position of a person being a distance computation target within an image, and an upward/downward orientation of the image capturing apparatus 20 that has generated the image. At this occasion, as will be described later in detail, the distance computation unit 13 uses a value (hereinafter, described as a reference height) set in advance as a height of a person.

Subsequently, the risk information generation unit 14 generates infection risk information by using the first distance generated in S30. The risk information generation unit 14 generates infection risk information, for example, based on any of the above-described methods 1 to 6. In a case of the example being real-time processing, the risk information generation unit 14 may adopt any of the methods 1, 3, 5, and 6, and evaluate a status at each time. Note that, the risk information generation unit 14 may adopt any of the methods 2, 4, and 6, and comprehensively evaluate whether a measure for suppressing spread of an infectious disease is appropriately taken within each target region, based on the entirety of a plurality of images generated for a predetermined time period up to the point of time.

Thereafter, the output unit 15 outputs infection risk information generated in S40 (S50). Then, thereafter, the image processing apparatus 10 repeats pieces of processing from S10 to S50.

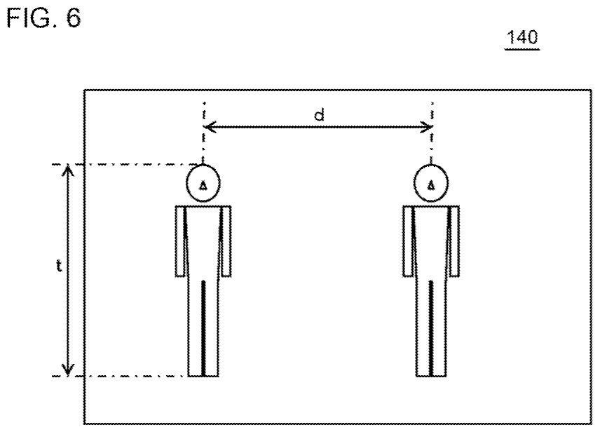

FIG. 6 is a diagram illustrating one example of a first distance computation method to be performed in step S30 in FIG. 5. The distance computation unit 13 determines a person as a reference. Further, the distance computation unit 13 performs processing illustrated in FIG. 6 for each of persons located around the person.

First, the distance computation unit 13 computes a height t of a person as a reference, or a person located around the person within an image. Herein, t is represented, for example, by the number of pixels. Subsequently, the distance computation unit 13 computes a distance d from the person as a reference to the person located around the person within the image. Herein, d is represented by the same unit (e.g., the number of pixels) as t. Subsequently, the distance computation unit 13 computes d/t, and computes a distance between the person as a reference and the person located around the person by multiplying the value d/t by the above-described reference height.

In a case where there is only one another person around a person as a reference, a distance computed for the another person becomes the first distance. Further, in a case where there are a plurality of other persons, the above-described distance is computed for each of these plurality of persons, and a minimum value of the distances becomes the first distance.

Note that, as described above, a reference height is set in advance. The reference height may be changed according to a place (e.g., a country) where the image capturing apparatus 20 is installed. For example, an average height of adults in a country where the target image capturing apparatus 20 is installed is used as the reference height. As a specific example of processing, the storage unit 16 stores information for determining a reference height, for each piece of image capturing apparatus identification information. Further, the distance computation unit 13 acquires image capturing apparatus identification information of the image capturing apparatus 20 that has generated an image being a processing target, and uses a reference height associated with the image capturing apparatus identification information by reading the reference height from the storage unit 16.

Further, in a case where an attribute (e.g., at least either one of gender and an age group) of a person being a computation target of the height t can be estimated by image processing, the distance computation unit 13 may change the reference height according to the attribute.

Note that, in most of images, a distortion specific to the image capturing apparatus 20 that has generated the image occurs. When computing the first distance, it is preferable that the distance computation unit 13 performs processing of correcting the distortion. The distance computation unit 13 performs distortion correction processing according to a position of a person within an image. Generally, a distortion of an image results from, for example, an optical system (e.g., a lens) included in the image capturing apparatus 20, and an upward/downward orientation (e.g., an angle with respect to a horizontal plane) of the image capturing apparatus 20. In view of the above, a content of distortion correction processing according to a position of a person within an image is set according to an optical system (e.g., a lens) included in the image capturing apparatus 20, and an upward/downward orientation of the image capturing apparatus 20.

Note that, in processing described by using FIG. 6, in a case where an object whose size is standardized to some extent is included in an image, the distance computation unit 13 may compute the first distance by using a size of the object, in place of a height of a person.

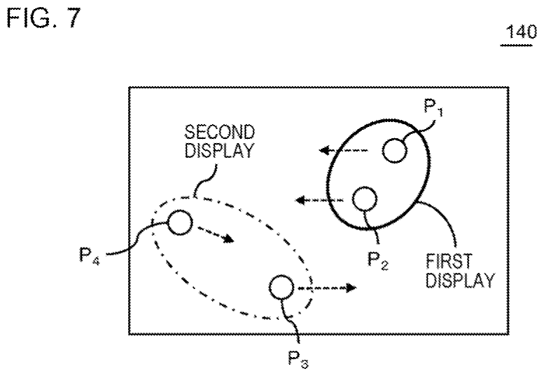

FIG. 7 is a diagram illustrating one example of an image to be displayed in step S40 in FIG. 5. The output is suitable for indicating a status at a point of time when each image is generated. In the example, the output unit 15 causes an output apparatus such as a display to display, together with infection risk information, an image (may be a moving image) used when the infection risk information is generated. At this occasion, the output unit 15 places, over an image, display for recognizing a combination of persons in which the first distance is equal to or less than a reference value, and then, causes to display the display and the image.

In the example illustrated in FIG. 7, the output unit 15 causes to display a mark indicating a combination of persons being a computation target of the first distance. Further, the output unit 15 changes a pattern of the mark according to determination as to whether the first distance is equal to or less than a reference value. In more detail, in the example illustrated in FIG. 7, two persons constituting a combination of persons are surrounded by a circle or an ellipse. Further, a display color and a pattern of a line (such as a solid line, a dotted line, and a dashed line) of the circle or the ellipse are changed according to determination as to whether the first distance is equal to or less than a reference value.

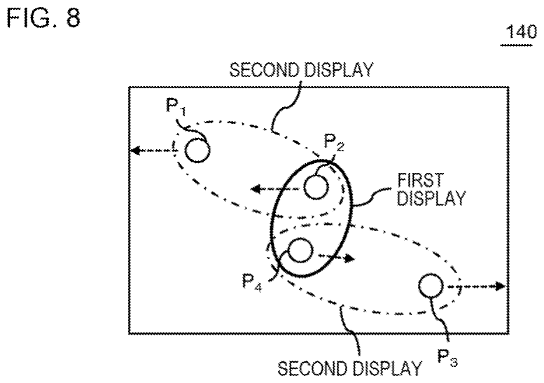

Note that, in a case where an image to be displayed is a moving image, as illustrated in FIGS. 7 and 8, as time elapses, a combination of persons being a computation target of the first distance changes. For example, at a timing in FIG. 7, a person P₁ becomes a partner for a person P₂ when the first distance is computed, but at a timing in FIG. 8 later than the timing, a person P₄ becomes a partner when the first distance is computed.

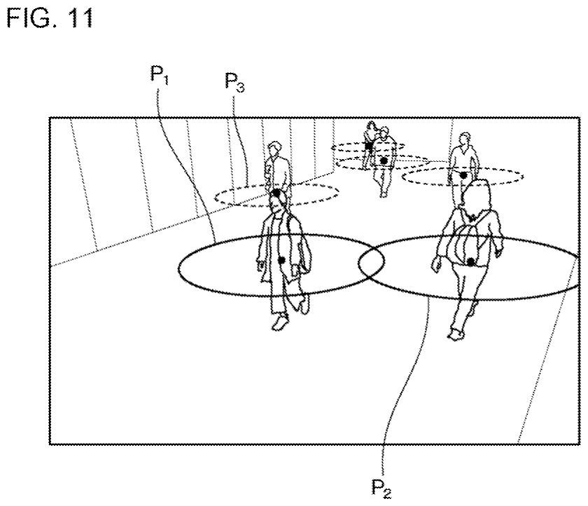

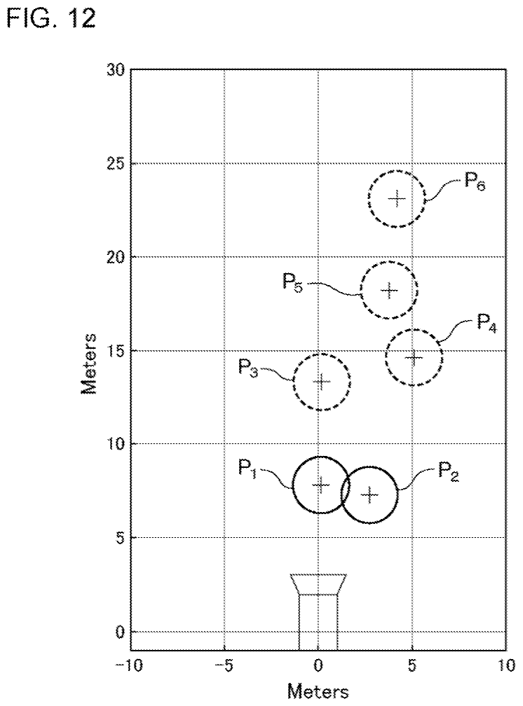

FIGS. 11 and 12 are diagrams illustrating one example of an image to be displayed on an output apparatus in step S40 in FIG. 5. In these drawings, the output unit 15 causes to display a mark indicating a range of a recommended value (e.g., the above-described reference value) of a social distance with respect to the person as a center, for each person. Further, in a case where a mark associated with a certain person and a mark associated with a person nearby the certain person overlap each other, specifically, in a case where a distance between a certain person and a person nearby the certain person (e.g., persons P₁ and P₂ in FIGS. 11 and 12) becomes equal to or less than a recommended value of a social distance, the output unit 15 causes to display a mark associated with each of these two persons in a pattern different from a mark (e.g., persons P₃, P₄, P₅, and P₆ in FIGS. 11 and 12) associated with another person. A way of differentiating a pattern includes, for example, changing a display color, differentiating a pattern of a line (such as a solid line, a dotted line, and a dash line) constituting a mark, and the like. In a case where a display color is changed, the output unit 15 displays, for example, a mark indicating a normal state in blue, and in a case where two marks overlap each other, these two marks are displayed in red.

In the example illustrated in FIG. 11, the output unit 15 places the above-described mark over an image (may be a moving image) used when infection risk information is generated. On the other hand, in the example illustrated in FIG. 12, the above-described mark overlaps a planar view, after a position of a person is illustrated in the planar view. The output unit 15 may display the display illustrated in FIG. 11 and the display illustrated in FIG. 12 simultaneously on an output apparatus.

Displays illustrated in FIGS. 7, 8, 11, and 12 may be performed by using a real-time moving image or image. In this case, displays illustrated in FIGS. 7, 8, 11, and 12 may be performed, for example, for an output apparatus such as a display installed near a target region, or may be used as contents of the Internet or broadcasting.

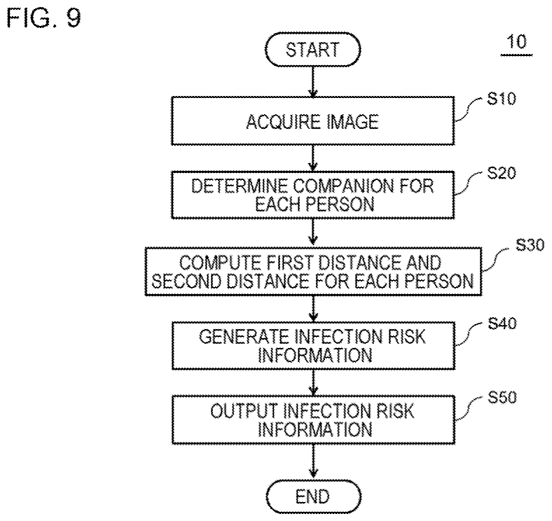

FIG. 9 is a flowchart illustrating a second example of processing to be performed by the image processing apparatus 10. In the example illustrated in FIG. 9, in S30, the distance computation unit 13 computes, in addition to the first distance, a second distance being a distance to a second nearest person among persons other than a companion, for each person. Further, in S40, the risk information generation unit 14 generates infection risk information by using the first distance and the second distance. The example is similar to the example illustrated in FIG. 5 except for this point.

The second distance is a distance between a person as a reference and a second nearest person to the person. A second distance computation method is similar to the first distance computation method except for a point that a distance to a second nearest person is selected, instead of a distance to a nearest person. Further, when infection risk information is generated, the risk information generation unit 14 increases a risk (lowers a safety rate), as the second distance decreases. Note that, the distance computation unit 13 may further generate a distance (third distance) between a person as a reference and a third nearest person to the person. In this case, the risk information generation unit 14 generates infection risk information, further with use of the third distance.

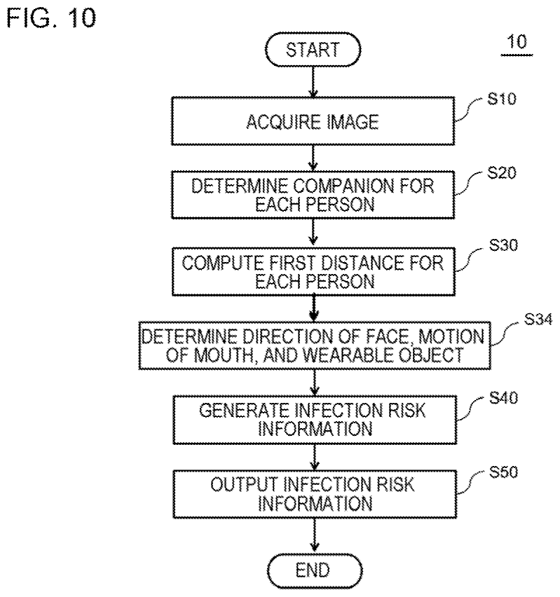

FIG. 10 is a flowchart illustrating a third example of processing to be performed by the image processing apparatus 10. The example illustrated in FIG. 10 is similar to the example illustrated in FIG. 5 or FIG. 9 except for a point that, when infection risk information is generated, the risk information generation unit 14 further uses information other than a person-to-person distance.

Specifically, S10 to S30 are similar to the example illustrated in FIG. 5 (or FIG. 9). Note that, in S30, the first distance and the second distance may be computed for each person. Further, by processing an image, the risk information generation unit 14 generates additional information necessary for generating infection risk information (S34). Information to be generated herein is at least one of a determination result on an orientation of a face of a person, a determination result on presence or absence of a wearable object for a face and a type of the wearable object, and a determination result on a motion of a mouth of a person.

“The orientation of a face of a person” at least includes at least either one of an orientation of a face of a person as a reference, and an orientation of a face of a nearest person to the person. Further, the risk information generation unit 14 increases a risk (lowers a safety rate) indicating infection risk information, as a face of a person approaches a direction facing a partner. Herein, in a case where the second distance or the third distance is used, the risk information generation unit 14 may further use an orientation of a face of a person being a partner when the second distance is computed, or an orientation of a face of a person being a partner when the third distance is computed.

“The presence or absence of a wearable object for a face” at least includes at least either one of presence or absence of a wearable object for a person as a reference, and presence or absence of a wearable object for a nearest person to the person. Further, in a case where a wearable object of a specific type is detected, the risk information generation unit 14 lowers a risk (raises a safety rate) indicating infection risk information, as compared with a case other than the above. Herein, a wearable object of a specific type is an object for covering at least either one of a mouth and a nose (preferably, both), for example, a mask or a muffler. Herein, in a case where the second distance or the third distance is used, the risk information generation unit 14 may further perform a similar operation for a person being a partner when the second distance is computed, or a person being a partner when the third distance is computed.

“The motion of a mouth” is that at least a mouth is moving. In a case where the mouth is moving, it is highly likely that the person is talking. In view of the above, in a case where a mouth is moving regarding at least either one of a person as a reference, and a nearest person to the person, the risk information generation unit 14 increases a risk (lowers a safety rate) indicating infection risk information, as compared with a case other than the above. Herein, in a case where the second distance or the third distance is used, the risk information generation unit 14 may further use a motion of a mouth of a person being a partner when the second distance is computed, or a motion of a mouth of a person being a partner when the third distance is computed.

As described above, according to the present example embodiment, by acquiring and processing an image generated by the image capturing apparatus 20, specifically, an image including a plurality of persons, the image processing apparatus 10 computes a distance (first distance) to a nearest person for the person regarding at least a part of the plurality of persons. The image processing apparatus 10 generates, with use of the first distance, infection risk information within a target region being a photographing target of the image capturing apparatus 20. Therefore, it is possible to recognize whether a measure for suppressing spread of an infectious disease is appropriately taken within a target region.

Meanwhile, it is difficult to thoroughly take a measure for securing a distance also for a group such as family members or lovers under authority of an administrator of each place. Therefore, it is not reasonable to evaluate that a measure for suppressing spread of an infectious disease is not appropriately taken at such a place, based on that a distance between companions is not secured. In contrast, the image processing apparatus 10 can generate infection risk information within each target region, without determining a companion by analyzing an image, and considering that a distance between companions is not secured. Consequently, it is possible to appropriately evaluate whether a measure for suppressing spread of an infectious disease is appropriately taken at each place, while suppressing the above-described inconvenience.

As described above, while the example embodiment according to the present invention has been described with reference to the drawings, the example embodiment is an example of the present invention, and various configurations other than the above can also be adopted.

Further, in a plurality of flowcharts used in the above description, a plurality of processes (pieces of processing) are described in order, but an order of execution of processes to be performed in each example embodiment is not limited to the order of description. In each example embodiment, the order of illustrated processes can be changed within a range that does not adversely affect a content. Further, the above-described example embodiments can be combined, as far as contents do not conflict with each other.

A part or all of the above-described example embodiment may also be described as the following supplementary notes, but is not limited to the following.

1. An image processing apparatus including:

a companion determination unit that determines a companion of each of a plurality of persons detected from an image, based on the image;

a distance computation unit that computes a first distance being a distance to a nearest person among the persons other than a companion, for the each person; and

a risk information generation unit that generates, with use of the first distance, infection risk information being information related to a risk of getting an infectious disease or a safety rate of not getting an infectious disease within a target region being a region included in the image.

2. The image processing apparatus according to supplementary note 1, wherein

the companion determination unit determines a companion of a first person, based on a duration of time of a state in which a distance between the first person and another person among a plurality of the persons is equal to or less than a threshold value.

3. The image processing apparatus according to supplementary note 1 or 2, wherein

the companion determination unit determines a companion of a first person, based on lines of sight of the first person and another person among a plurality of the persons.

4. The image processing apparatus according to supplementary note 3, wherein

the companion determination unit determines a companion of the first person, based on at least either one of a duration of time of a line-of-sight state in which lines of sight of the first person and the another person are directed toward each other, and a frequency of occurrence of the line-of-sight state within a predetermined time.

5. The image processing apparatus according to any one of supplementary notes 1 to 4, wherein

the companion determination unit determines a companion of a first person, based on a physical contact status of the first person and another person among a plurality of the persons.

6. The image processing apparatus according to any one of supplementary notes 2 to 5, wherein

the companion determination unit determines a companion of a first person, based on attributes of the first person and another person among a plurality of the persons.

7. The image processing apparatus according to any one of supplementary notes 1 to 6, wherein

the distance computation unit computes a second distance being a distance to a second nearest person among the persons other than a companion, for the each person, and

the risk information generation unit generates the infection risk information, further with use of the second distance.

8. An image processing method including,

by a computer:

determining a companion of each of a plurality of persons detected from an image, based on the image;

computing a first distance being a distance to a nearest person among the persons other than a companion, for the each person; and

generating, with use of the first distance, infection risk information being information related to a risk of getting an infectious disease or a safety rate of not getting an infectious disease within a target region being a region included in the image.

9. A program causing a computer to function as:

a companion determination unit that determines a companion of each of a plurality of persons detected from an image, based on the image;

a distance computation unit that computes a first distance being a distance to a nearest person among the persons other than a companion, for the each person; and

a risk information generation unit that generates, with use of the first distance, infection risk information being information related to a risk of getting an infectious disease or a safety rate of not getting an infectious disease within a target region being a region included in the image.

This application is based upon and claims the benefit of priority from Japanese patent application No. 2020-098403, filed on Jun. 5, 2020, the disclosure of which is incorporated herein in its entirety by reference.

### REFERENCE SIGNS LIST

10 Image processing apparatus
11 Image acquisition unit
12 Companion determination unit
13 Distance computation unit
14 Risk information generation unit
15 Output unit
16 Storage unit
20 Image capturing apparatus

What is claimed is:

1. An image processing apparatus comprising:
at least one memory configured to store one or more instructions; and
at least one processor configured to execute the one or more instructions to:
determine a companion of each of a plurality of persons detected from an image, based on the image;
compute a first distance being a distance to a nearest person among the persons other than a companion, for the each person; and
generate, with use of the first distance, infection risk information being information related to a risk of getting an infectious disease or a safety rate of not getting an infectious disease within a target region being a region included in the image.

2. The image processing apparatus according to claim 1, wherein
the processor is further configured to execute the one or more instructions to determine a companion of a first person, based on a duration of time of a state in which a distance between the first person and another person among a plurality of the persons is equal to or less than a threshold value.

3. The image processing apparatus according to claim 1, wherein
the processor is further configured to execute the one or more instructions to determine a companion of a first person, based on lines of sight of the first person and another person among a plurality of the persons.

4. The image processing apparatus according to claim 3, wherein
the processor is further configured to execute the one or more instructions to determine a companion of the first person, based on at least either one of a duration of time of a line-of-sight state in which lines of sight of the first person and the another person are directed toward each other, and a frequency of occurrence of the line-of-sight state within a predetermined time.

5. The image processing apparatus according to claim 1, wherein
the processor is further configured to execute the one or more instructions to determine a companion of a first person, based on a physical contact status of the first person and another person among a plurality of the persons.

6. The image processing apparatus according to claim 2, wherein
the processor is further configured to execute the one or more instructions to determine a companion of a first person, based on attributes of the first person and another person among a plurality of the persons.

7. The image processing apparatus according to claim 1, wherein the processor is further configured to execute the one or more instructions to:
compute a second distance being a distance to a second nearest person among the persons other than a companion, for the each person, and
generate the infection risk information, further with use of the second distance.

8. An image processing method comprising,
by a computer:
determining a companion of each of a plurality of persons detected from an image, based on the image;
computing a first distance being a distance to a nearest person among the persons other than a companion, for the each person; and
generating, with use of the first distance, infection risk information being information related to a risk of getting an infectious disease or a safety rate of not getting an infectious disease within a target region being a region included in the image.

9. A non-transitory storage medium storing a program causing a computer to:
determine a companion of each of a plurality of persons detected from an image, based on the image;
compute a first distance being a distance to a nearest person among the persons other than a companion, for the each person; and
generate, with use of the first distance, infection risk information being information related to a risk of getting an infectious disease or a safety rate of not getting an infectious disease within a target region being a region included in the image.
